(19) United States
(12) Patent Application Publication
(10) Pub. No.: US 2026/0087616 A1
(43) Pub. Date: Mar. 26, 2026

(54) METHOD AND APPARATUS FOR PROCESSING IMAGE DATA IN A MEDICAL IMAGING SYSTEM

(71) Applicant: CANON MEDICAL SYSTEMS CORPORATION, Tochigi (JP)

(72) Inventors: Joseph WHITEHEAD, Vernon Hills, IL (US); Joseph MANAK, Vernon Hills, IL (US); Yi HU, Vernon Hills, IL (US)

(73) Assignee: CANON MEDICAL SYSTEMS CORPORATION, Tochigi (JP)

(21) Appl. No.: 18/897,748

(22) Filed: Sep. 26, 2024

(51) Int. Cl. G06T 7/00; G16H 30/40

(52) U.S. Cl. CPC G06T 7/0012; G16H 30/40; G06T 2207/20081; G06T 2207/20084; G06T 2207/30004; G06T 2207/30168

(57) ABSTRACT

A method for performing image data processing in a medical imaging system is provided. The method includes collecting a first image dataset, using the collected first image dataset, training a first neural network, based on a loss function having a perceptual component; and using the trained first neural network, inferring output image data from input image data obtained by the medical imaging system, such that the inferred output image data has an image quality better than an image quality of the obtained input image data. The training of the first neural network uses a pretrained second neural network. The pretrained second neural network is specific to a particular domain to which the medical imaging system corresponds.

### BACKGROUND

### Field

This disclosure relates to medical imaging techniques, including, but not limited to 2D projection X-ray imaging, Computed Tomography (CT) imaging, Magnetic Resonance Imaging (MRI), Positron Emission Tomography (PET) imaging, and ultrasound (US) imaging.

### Description of the Related Art

Deep-learning-based image data processing approaches have proven to out-perform classical methods for various medical imaging modalities. However, these approaches often suffer from image blurs in the final output images. To reduce image blurs, advanced techniques such as perceptual loss and contrastive learning loss have been proposed for training neural networks. In a general sense, these techniques use an encoder to extract image features. During network training, the difference between the features extracted from the network's output and the features extracted from the target image is minimized, with the goal of preserving the features extracted by the encoder.

Typically, the encoder is implemented by another neural network, such as VGG19 and VGG16. However, these neural networks are generally trained on millions of natural images for classification purposes, not specifically for a medical imaging domain. As a result, the extracted features may not be relevant to the medical imaging tasks.

There is a need for improved approaches that provide more domain-specific and task-relevant training of neural networks used in medical imaging systems to enhance the image quality

### SUMMARY

The present disclosure relates to a method for performing image data processing in a medical imaging system. The method includes collecting a first image dataset, using the collected first image dataset, training a first neural network, based on a loss function having a perceptual component; and using the trained first neural network, inferring output image data from input image data obtained by the medical imaging system, such that the inferred output image data has an image quality better than an image quality of the obtained input image data. The training of the first neural network uses a pretrained second neural network. The pretrained second neural network is specific to a particular domain to which the medical imaging system corresponds.

The disclosure additionally relates to an apparatus for performing image data processing in a medical imaging system. The apparatus includes processing circuitry configured to: collect a first image dataset, using the collected first image dataset, train a first neural network, based on a loss function having a perceptual component, and using the trained first neural network, infer output image data from input image data obtained by the medical imaging system, such that the inferred output image data has an image quality better than an image quality of the obtained input image data. The training of the first neural network uses a pretrained second neural network. The pretrained second neural network is specific to a particular domain to which the medical imaging system corresponds.

The disclosure also relates to a non-transitory computer-readable medium storing instructions. The instructions, when executed by a processor, can cause the processor to perform the above method for performing image data processing in a medical imaging system.

Note that this summary section does not specify every embodiment and/or incrementally novel aspect of the present disclosure or claimed invention. Instead, the summary only provides a preliminary discussion of different embodiments and corresponding points of novelty. For additional details and/or possible perspectives of the invention and embodiments, the reader is directed to the Detailed Description section and corresponding figures of the present disclosure as further discussed below.

### BRIEF DESCRIPTION OF THE DRAWINGS

Various embodiments of this disclosure that are proposed as examples will be described in detail with reference to the following figures, wherein like numerals reference like elements, and wherein:

FIG. 1 shows examples of different medical imaging domains in accordance with embodiments of the disclosure;

FIG. 2 shows an exemplary scenario of training a denoising neural network, based on a contrastive learning loss function;

FIG. 3 shows an exemplary scenario of training a denoising neural network, based on a perceptual loss function;

FIG. 4 shows a block diagram of an imaging data processing apparatus 400 in accordance with embodiments of the disclosure;

FIG. 5 shows an exemplary scenario of using a pre-trained domain-specific feature extractor to train a denoising neural network, based on a contrastive learning loss function, in accordance with embodiments of the disclosure;

FIG. 6 shows an exemplary scenario of using a pre-trained domain-specific feature extractor to train a denoising neural network, based on a perceptual loss function, in accordance with embodiments of the disclosure;

FIG. 7 shows an exemplary scenario where a generative adversarial network is trained to obtain a domain-specific feature extractor (implemented by the discriminator portion of the generative adversarial network), in accordance with embodiments of the disclosure;

FIG. 8 shows a flow chart of an exemplary procedure 800 for performing image data processing in accordance with embodiments of the disclosure;

FIG. 9 shows a schematic block diagram of an exemplary X-ray diagnostic system that can incorporate the techniques disclosed herein;

FIG. 10 is a schematic of an implementation of an exemplary computed tomography (CT) scanner; and

FIG. 11 is a block diagram illustrating an exemplary computer system for implementing the machine learning training and inference methods according to an exemplary aspect of the disclosure.

### DETAILED DESCRIPTION

The following disclosure provides embodiments or examples for implementing different features of the provided subject matter. Specific examples of components and arrangements are described below to simplify the present disclosure. These are, of course, merely examples and are not intended to be limiting.

For example, the order of discussion of the different steps as described herein has been presented for the sake of clarity. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present invention can be embodied and viewed in many different ways.

Furthermore, as used herein, the words “a,” “an,” and the like generally carry a meaning of “one or more,”unless stated otherwise.

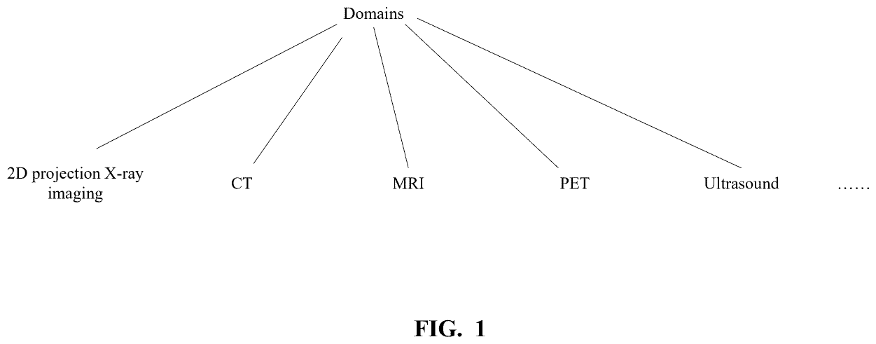

Neural networks have been used across various medical imaging modalities to enhance the image quality. FIG. 1 shows examples of different medical imaging domains in accordance with embodiments of the disclosure. These domains include, but are not limited to, 2D projection X-ray imaging, Computed Tomography (CT), Magnetic Resonance Imaging (MRI), Positron Emission Tomography (PET), ultrasound (US), etc. Each medical imaging modality can have its unique characteristics. For example, compared with CT or US images, MRI images may have different texture patterns and noise characteristics.

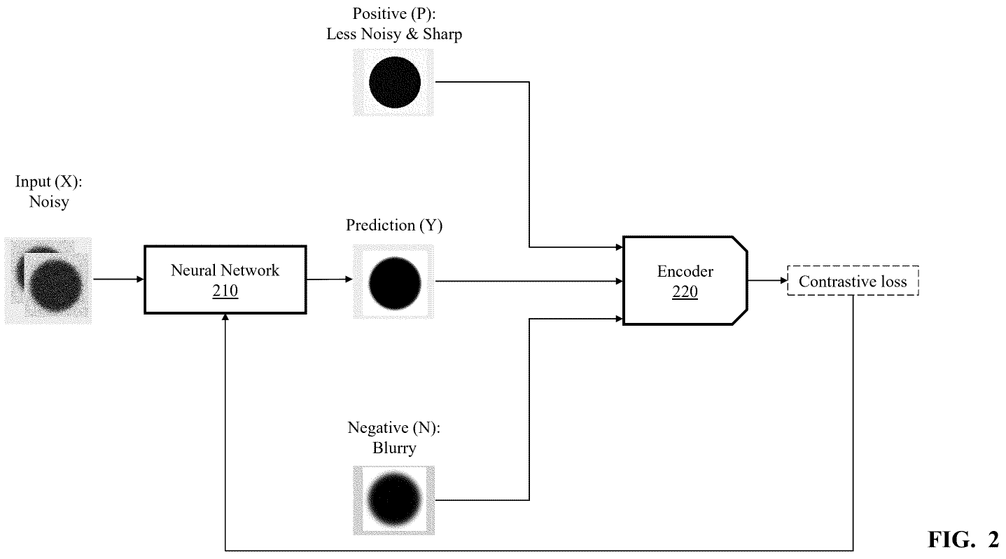

To suppress image blurs in the final images inferred by neural networks, advanced training techniques such as perceptual loss and contrastive learning have been used. FIG. 2 shows an exemplary scenario of training a denoising neural network based on a contrastive learning loss function. In contrastive learning, both a positive target (P) and a negative target (N) are provided. The network 210 learns to take an input image (X) and produce a predicted image (Y) resembling the positive target (P), which is less noisy and sharper, while avoiding the negative target (N), which may be less noisy but blurry, for example. This is achieved by first extracting features from both the positive and negative targets (P, N) through an encoder 220. Then, the network 210 learns to keep the features extracted from the positive target (P) and avoid the features extracted from the negative target (N). Alternatively, the network 210 can only learn to maintain specific features extracted from the positive target (P), or only learn to avoid specific features extracted from the negative target (N).

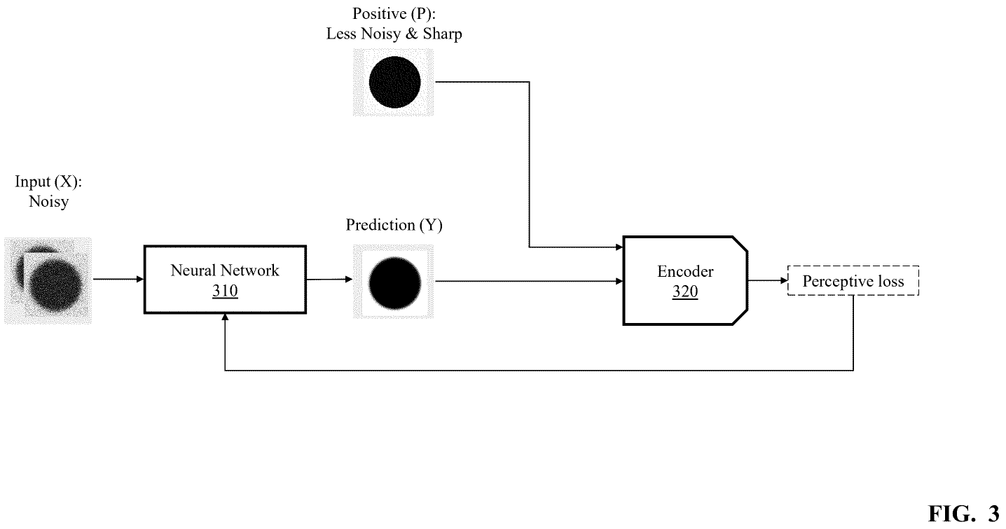

FIG. 3 shows an exemplary scenario of training a denoising neural network based on a perceptual loss function. This perceptual loss approach is similar to the contrastive learning approach, except no negative sample is provided. The network 310 learns to take an input image (X) and produce a predicted image (Y) resembling a positive target (P). Features are extracted by an encoder 320 from the positive target (P). Then, the network 310 learns to maintain the features extracted from the positive target (P).

The encoders 220 and 320 are usually neural networks pre-trained using a large number of natural images (e.g., photographs) for classification purposes, such as VGG19 and VGG16. Generally, it is difficult and time-consuming to have a domain expert (e.g., a radiologist with the expertise in the specific medical imaging domain) hand-craft features for the perceptual loss or contrastive learning loss. Additionally, it is impractical for a domain expert to be available during network training to vote on the image quality at each iteration. Thus, the encoders 220 and 320 are not domain-specific and typically not an expert in the specific medical imaging domain.

The present disclosure provides a method and apparatus for performing deep-learning-based image data processing in medical imaging systems. A neural network for improving the image quality is trained based on a loss function with a perceptual component. By pre-training a generative adversarial network (GAN), a feature extractor can learn to identify and extract relevant features specific to the domain of the medical imaging system. This pre-trained feature extractor can serve as a domain expert, and can be used in the perceptual component of the loss function.

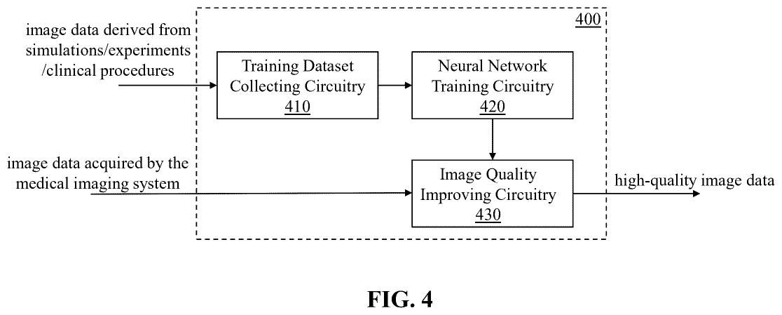

FIG. 4 shows a block diagram of an imaging data processing apparatus 400 in accordance with embodiments of the disclosure. The imaging data processing apparatus 400 includes training dataset collecting circuitry 410, neural network training circuitry 420, and image quality improving circuitry 430.

The training dataset collecting circuitry 410 can gather image data to create a dataset for training a neural network aimed at improving the image quality in the medical imaging system. The image data can be data collected from the domain of the medical imaging system. For instance, the training dataset can include image data generated through physical simulations, obtained from research experiments on phantoms and volunteers, and acquired during clinical procedures on patients, etc.

The neural network training circuitry 420 uses the training dataset collected by the training dataset collecting circuitry 410 to train the neural network. The neural network can be a denoising network, a deblurring network, or a network trained to remove artifacts in images, for example.

Once the network parameters are determined through training, the neural network can serve as the image quality improving circuitry 430. It receives, as an input, image data acquired by the medical imaging system, which typically has a low image quality, and infers image data with a high image quality at its output.

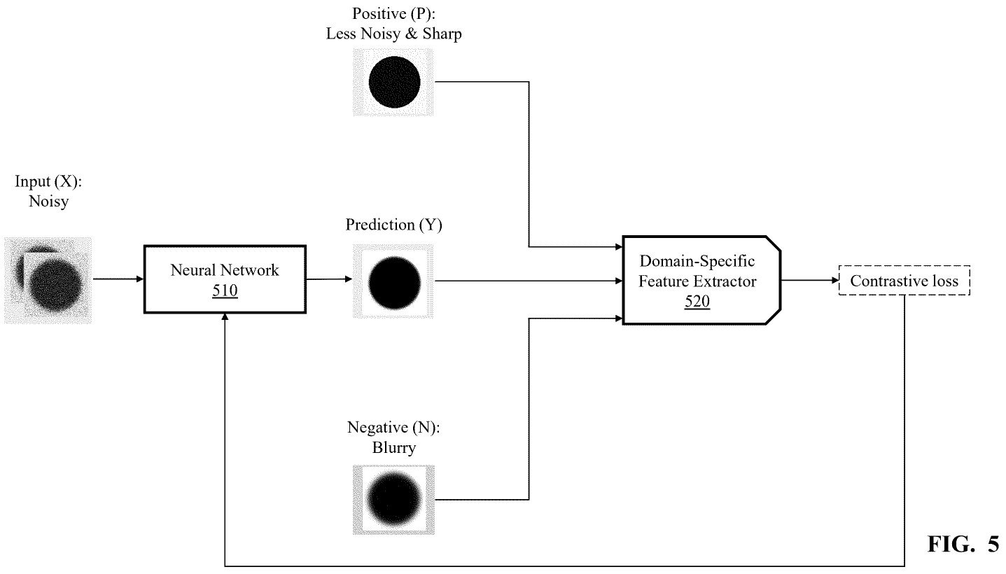

FIG. 5 shows an exemplary scenario for training a denoising neural network 510, based on a contrastive learning loss function, in accordance with embodiments of the disclosure. A feature extractor 520 is used in a perceptive component of the contrastive learning loss function. This feature extractor 520 is pre-trained to function as a domain expert.

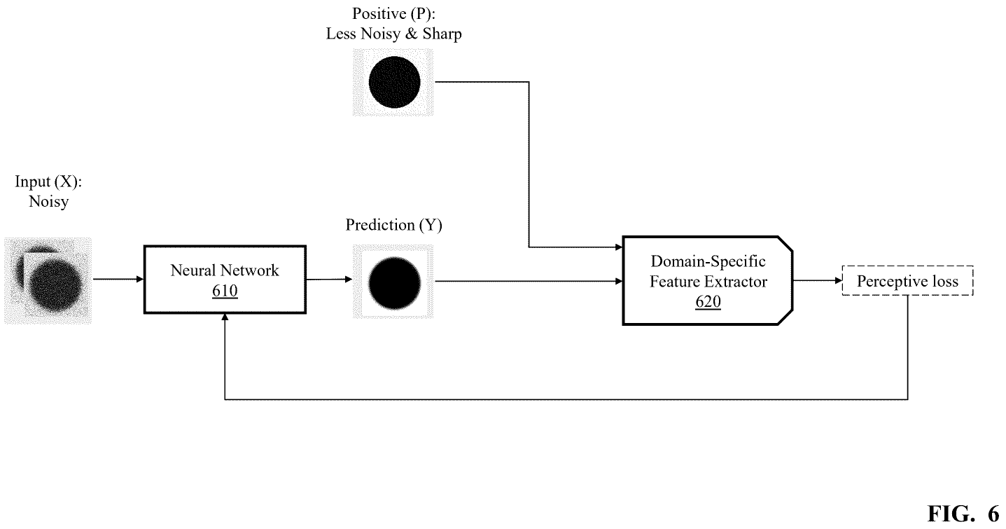

Similarly, in the exemplary scenario shown in FIG. 6 where a denoising neural network 610 is trained based on a perceptive loss function, a feature extractor 620 is included in a perceptive component of the perceptive loss. Like the feature extractor 520, this feature extractor 620 is also pre-trained as a domain expert. Although not shown in FIG. 5 or FIG. 6, both the contrastive learning loss function and the contrastive learning loss function can be paired with an additional loss function that does not use perception, e.g., mean squared error.

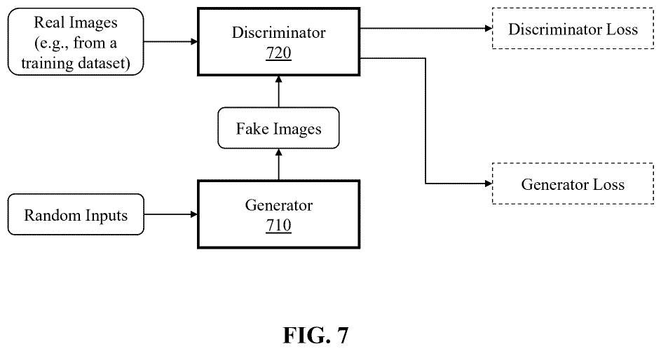

The feature extractors 520 and 620 can be implemented by the discriminator portion of a pre-trained GAN. FIG. 7 shows an exemplary scenario for training a GAN to obtain a domain-specific feature extractor, in accordance with embodiments of the disclosure.

As shown in FIG. 7, the GAN includes a generator 710 and a discriminator 720. The generator 710 generates images with the goal of fooling the discriminator 720 into believing that the generated images are real. The discriminator 720 evaluates the images generated by the generator 710 and determines whether they are machine-generated or real images from a training dataset, attempting to identify the fake images.

For example, the generator 710 can take random vectors as input. Typically, these random inputs follow a multi-dimensional Gaussian distribution. The incorporation of randomness into the model prevents it from merely memorizing the training data. Instead, the generator 710 maps the random inputs to new images that are not present in the training dataset. Through adversarial training, the generator 710 learns to close the distribution gap between these generated images and real images, making the generated images highly realistic. The discriminator 720 learns to classify images based on their authenticity, assigning a score (e.g., 0) for the generated images, and a different score (e.g., 1) for real images from the training dataset. To successfully differentiate between real and synthetic images, the discriminator 720 must learn high-level, domain-specific features of the training dataset, thereby developing a comprehensive understanding of what features are critical in the specific domain.

During the GAN training process, the generator 710 and the discriminator 720 are trained alternately in an iterative manner. At the beginning, the generator 710 may produce images that are easily identified as fake by the discriminator 720. However, with continuous training, the generator 710 improves its ability to create convincing images, eventually producing images that can deceive the discriminator 720. At this stage, the discriminator 720's ability to distinguish between real and generated images may diminish.

Once the generator 710 has become sufficiently adept at deceiving the discriminator 720, further training of the generator 710 is no longer beneficial. At this stage, the parameters of the generator 710 are fixed, and the training of the discriminator 720 begins. Through continuous training, the discriminator 720 enhances its ability to accurately identify fake images. When the discriminator 720 achieves a high level of proficiency, the generator 710 can no longer deceive it.

By iterating through these two training phases, both the generator 710 and the discriminator 720 continually improve their abilities. The generator 710 becomes better at creating realistic images, while the discriminator 720 becomes more skilled at identifying fake images. Once the GAN is trained, the discriminator portion of the GAN can be used as the feature extractors 520 and 620.

There are no specific limitations to the loss functions or network architectures of the generator 710 or the discriminator 720. The discriminator portion can be any model suitable for the specific imaging task. For example, a U-net can be used, and the extracted features can be outputs from a single layer or a combination of multiple layers within the trained U-net.

The training dataset for the GAN can be gathered from simulations, experiments, and/or clinical procedures in the specific medical imaging domain. In one embodiment, the GAN can be trained using the same training dataset prepared for training the desired neural network aimed at improving the image quality. For instance, in the examples shown in FIGS. 5 and 6, training a denoising network may require pairs of clean and noisy image data. Accordingly, the clean and/or noisy image data can be used to train the GAN.

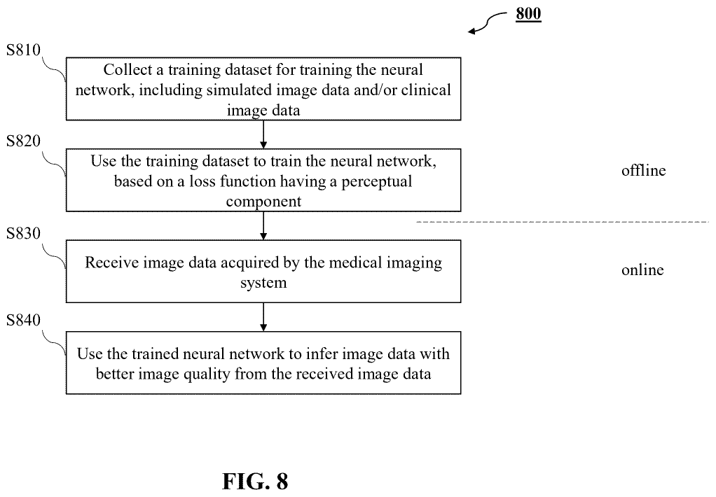

FIG. 8 shows a flow chart of an exemplary procedure 800 for performing image data processing in accordance with embodiments of the disclosure. The procedure 800 includes an offline portion (steps S810 and S820) and an online portion (steps S830 and S840). In step S810, a training dataset is collected for training the neural network aimed at improving the image quality. In step S820, the neural network is trained using the training dataset, based on a loss function with a perceptual component. A feature extractor is pre-trained as a domain expert and used in the perceptive component of the perceptive loss.

In step S830, image data acquired by the medical imaging system is received. In step S840, the trained neural network is used to infer higher-quality image data from the received lower-quality image data. As the perceptual component of the loss function extracts relevant features specific to the particular imaging domain, the final output images are pushed to resemble the domain-specific features rather than arbitrary features extracted from networks trained to perform other tasks.

This approach can be used in any imaging domain where adequate samples of real data can be collected to train a GAN. Although the present disclosure is described and illustrated to train a denoising neural network based on a contrastive learning loss function or a perceptual loss function, one of skills in the field can recognize that any forms of loss function that has a perceptual component can be used.

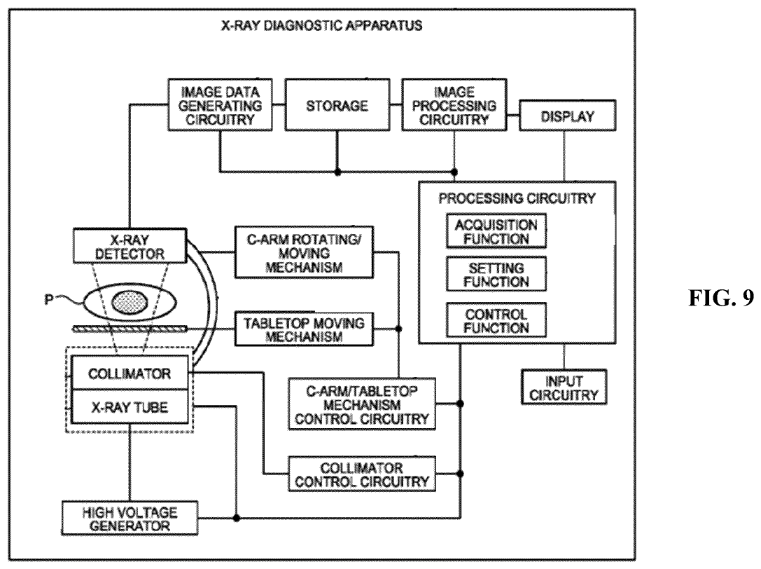

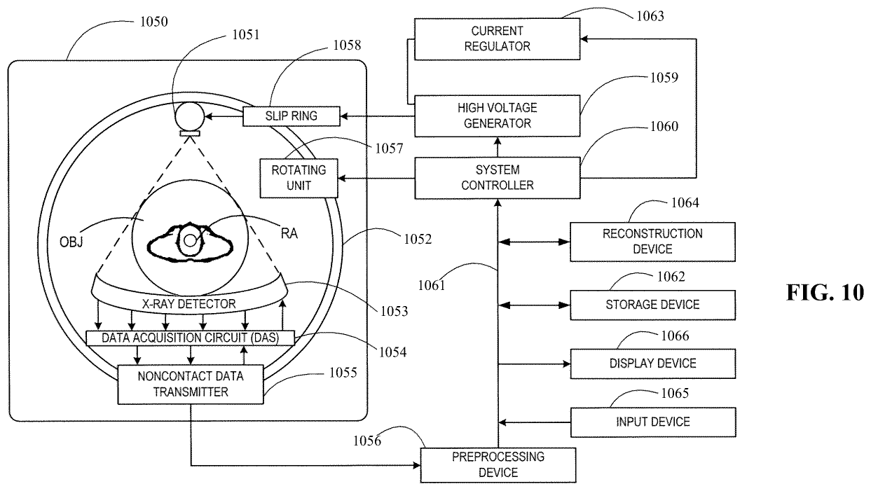

FIG. 9 shows a schematic block diagram of an exemplary X-ray diagnostic system that can incorporate the techniques disclosed herein. FIG. 10 provides a schematic of an implementation of an exemplary CT scanner. This approach can be applied to any imaging modality, including, but not limited to, 2D projection X-ray imaging, CT, MRI, PET, US, etc.

As shown in FIG. 10, a radiography gantry 1050 is illustrated from a side view and further includes an X-ray tube 1051, an annular frame 1052, and a multi-row or two-dimensional-array-type X-ray detector 1053. The X-ray tube 1051 and X-ray detector 1053 are diametrically mounted across an object OBJ on the annular frame 1052, which is rotatably supported around a rotation axis RA. A rotating unit 1057 rotates the annular frame 1052 at a high speed, such as 0.4 sec/rotation, while the object OBJ is being moved along the axis RA into or out of the illustrated page.

An embodiment of an X-ray CT apparatus according to the present disclosure will be described below with reference to the views of the accompanying drawing. Note that X-ray CT apparatuses include various types of apparatuses, e.g., a rotate/rotate-type apparatus in which an X-ray tube and X-ray detector rotate together around an object to be examined, and a stationary/rotate-type apparatus in which many detection elements are arrayed in the form of a ring or plane, and only an X-ray tube rotates around an object to be examined. The present disclosure can be applied to either type. In this case, the rotate/rotate-type, which is currently the mainstream, will be exemplified.

The multi-slice X-ray CT apparatus further includes a high voltage generator 1059 that generates a tube voltage applied to the X-ray tube 1051 through a slip ring 1058 so that the X-ray tube 1051 generates X-rays. The X-rays are emitted towards the object OBJ, whose cross-sectional area is represented by a circle. For example, the X-ray tube 1051 having an average X-ray energy during a first scan that is less than an average X-ray energy during a second scan. Thus, two or more scans can be obtained corresponding to different X-ray energies. The X-ray detector 1053 is located at the opposite side from the X-ray tube 1051 across the object OBJ for detecting the emitted X-rays that have transmitted through the object OBJ. The X-ray detector 1053 further includes individual detector elements or units.

The CT apparatus further includes other devices for processing the detected signals from the X-ray detector 1053. A data acquisition circuit or a Data Acquisition System (DAS) 1054 converts a signal output from the X-ray detector 1053 for each channel into a voltage signal, amplifies the signal, and further converts the signal into a digital signal. The X-ray detector 1053 and the DAS 1054 are configured to handle a predetermined total number of projections per rotation (TPPR).

The above-described data is sent to a preprocessing device 1056, which is housed in a console outside the radiography gantry 1050 through a non-contact data transmitter 1055. The preprocessing device 1056 performs certain corrections, such as sensitivity correction, on the raw data. A memory 1062 stores the resultant data, which is also called projection data at a stage immediately before reconstruction processing. The memory 1062 is connected to a system controller 1060 through a data/control bus 1061, together with a reconstruction device 1064, input device 1065, and display 1066. The system controller 1060 controls a current regulator 1063 that limits the current to a level sufficient for driving the CT system.

The detectors are rotated and/or fixed with respect to the patient among various generations of the CT scanner systems. In one implementation, the above-described CT system can be an example of a combined third-generation geometry and fourth-generation geometry system. In the third-generation system, the X-ray tube 1051 and the X-ray detector 1053 are diametrically mounted on the annular frame 1052 and are rotated around the object OBJ as the annular frame 1052 is rotated about the rotation axis RA. In the fourth-generation geometry system, the detectors are fixedly placed around the patient and an X-ray tube rotates around the patient. In an alternative embodiment, the radiography gantry 1050 has multiple detectors arranged on the annular frame 1052, which is supported by a C-arm and a stand.

The memory 1062 can store the measurement value representative of the irradiance of the X-rays at the X-ray detector unit 1053. Further, the memory 1062 can store a dedicated program for executing the CT image reconstruction, material decomposition, and motion estimation and motion compensation methods including the methods described herein.

The reconstruction device 1064 can execute the above-referenced methods, described herein. Further, reconstruction device 1064 can execute pre-reconstruction processing image processing such as volume rendering processing and image difference processing as needed.

The pre-reconstruction processing of the projection data performed by the preprocessing device 1056 can include correcting for detector calibrations, detector nonlinearities, and polar effects, for example.

Post-reconstruction processing performed by the reconstruction device 1064 can include filtering and smoothing the image, volume rendering processing, and image difference processing, as needed. The image reconstruction process can be performed using filtered back projection, iterative image reconstruction methods, or stochastic image reconstruction methods. The reconstruction device 1064 can use the memory to store, e.g., projection data, reconstructed images, calibration data and parameters, and computer programs.

The reconstruction device 1064 can include a CPU (processing circuitry) that can be implemented as discrete logic gates, as an Application Specific Integrated Circuit (ASIC), a Field Programmable Gate Array (FPGA) or other Complex Programmable Logic Device (CPLD). An FPGA or CPLD implementation may be coded in VDHL, Verilog, or any other hardware description language and the code may be stored in an electronic memory directly within the FPGA or CPLD, or as a separate electronic memory. Further, the memory 1062 can be non-volatile, such as ROM, EPROM, EEPROM or FLASH memory. The memory 1062 can also be volatile, such as static or dynamic RAM, and a processor, such as a microcontroller or microprocessor, can be provided to manage the electronic memory as well as the interaction between the FPGA or CPLD and the memory.

Alternatively, the CPU in the reconstruction device 1064 can execute a computer program including a set of computer-readable instructions that perform the functions described herein, the program being stored in any of the above-described non-transitory electronic memories and/or a hard disc drive, CD, DVD, FLASH drive or any other known storage media. Further, the computer-readable instructions may be provided as a utility application, background daemon, or component of an operating system, or combination thereof, executing in conjunction with a processor, such as a Xeon processor from Intel of America or an Opteron processor from AMD of America and an operating system, such as Microsoft 10, UNIX, Solaris, LINUX, Apple, MAC-OS and other operating systems known to those skilled in the art. Further, CPU can be implemented as multiple processors cooperatively working in parallel to perform the instructions.

In one implementation, the reconstructed images can be displayed on a display 1066. The display 1066 can be an LCD display, CRT display, plasma display, OLED, LED or any other display known in the art.

The memory 1062 can be a hard disk drive, CD-ROM drive, DVD drive, FLASH drive, RAM, ROM or any other electronic storage known in the art.

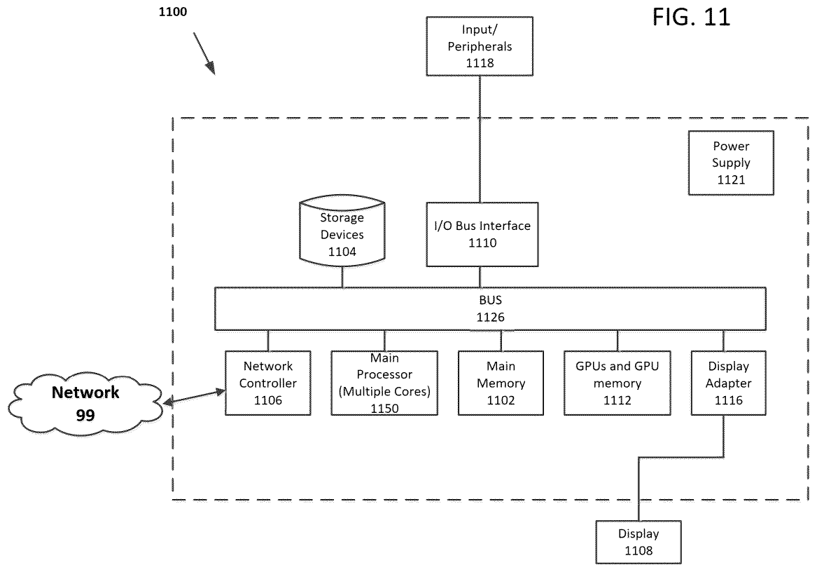

FIG. 11 is a block diagram illustrating an example computer system for implementing the machine learning training and inference methods according to an exemplary aspect of the disclosure. In a non-limiting example, the computer system can be an AI workstation running an operating system, for example Ubuntu Linux OS, Windows, a version of Unix OS, or Mac OS. The computer system 1100 can include one or more central processing units (CPU) 1150 having multiple cores. The computer system 1100 can include a graphics board 1112 having multiple GPUs, each GPU having GPU memory. The graphics board 1112 can perform many of the mathematical operations of the disclosed machine learning methods. The computer system 1100 includes main memory 1102, typically random access memory RAM, which contains the software being executed by the processing cores 1150 and GPUs 1112, as well as a non-volatile storage device 1104 for storing data and the software programs. Several interfaces for interacting with the computer system 1100 may be provided, including an I/O Bus Interface 1110, Input/Peripherals 1118 such as a keyboard, touch pad, mouse, Display Adapter 1116 and one or more Displays 1108, and a Network Controller 1106 to enable wired or wireless communication through a network 99. The interfaces, memory and processors may communicate over the system bus 1126. The computer system 1100 includes a power supply 1121, which may be a redundant power supply.

In one embodiment, the computer system 1100 includes a multicore CPU and a graphics card by NVIDIA, in which the GPUs have multiple cores. In one embodiment, the computer system 1100 may include a machine learning engine 1112.

Numerous modifications and variations of the embodiments presented herein are possible in light of the above teachings. It is therefore to be understood that within the scope of the claims, the application may be practiced otherwise than as specifically described herein. The inventions are not limited to the examples that have just been described; it is in particular possible to combine features of the illustrated examples with one another in variants that have not been illustrated.

Embodiments of the present disclosure may also be as set forth in the following parentheticals.
(1) A method for performing image data processing in a medical imaging system, the method comprising: collecting a first image dataset; using the collected first image dataset, training a first neural network, based on a loss function having a perceptual component; and using the trained first neural network, inferring output image data from input image data obtained by the medical imaging system, such that the inferred output image data has an image quality better than an image quality of the obtained input image data, wherein the training of the first neural network uses a pretrained second neural network, and the pretrained second neural network is specific to a particular domain to which the medical imaging system corresponds.
(2) The method of (1), further comprising: obtaining a second image dataset, and using the obtained second image dataset to train, as the pretrained second neural network, a feature extractor for extracting a feature specific to the particular domain.
(3) The method of (2), wherein the step of training the feature extractor further comprises: iteratively alternating between training a generator included in a generative adversarial neural network and a discriminator included in the generative adversarial neural network, until a predetermined criterion is met, and using the trained discriminator as the pretrained second neural network.
(4) The method of (3), wherein the discriminator includes one or more layers of a U-net.
(5) The method of (1), wherein the collecting step further comprises collecting the first image dataset through a simulation, an experiment, and/or a clinical procedure within the particular domain.
(6) The method of (2), wherein the obtaining step further comprises obtaining the second image dataset through a simulation, an experiment, and/or a clinical procedure within the particular domain.
(7) The method of (5), wherein the obtaining step further comprises using the collected first image dataset, or a subset of the collected first image dataset, as the obtained second image dataset.
(8) The method of (1), wherein the loss function is a contrastive learning loss function, and the step of training the first neural network further comprises: obtaining, from the collected first image dataset, first image data, second image data, and third image data, and based on the contrastive learning loss function, using the first image data as input data, and the second and third image data as label data, to update a parameter of the first neural network, until a predetermined criterion is met.
(9) The method of (1), wherein the loss function is a perceptual loss function, and the step of training the first neural network further comprises: obtaining, from the collected first image dataset, first image data and second image data, and based on the perceptual loss function, using the first image data as input data and the second image data as label data to update a parameter of the first neural network, until a predetermined criterion is met.
(10) The method of (1), wherein the particular domain is 2D projection X-ray imaging, Computed Tomography (CT) imaging, Magnetic Resonance Imaging (MRI), Positron Emission Tomography (PET) imaging, or ultrasound (US).
(11) An apparatus for performing image data processing in a medical imaging system, the apparatus comprising: processing circuitry configured to collect a first image dataset, using the collected first image dataset, train a first neural network, based on a loss function having a perceptual component, and using the trained first neural network, infer output image data from input image data obtained by the medical imaging system, such that the inferred output image data has an image quality better than an image quality of the obtained input image data, wherein the training of the first neural network uses a pretrained second neural network, and the pretrained second neural network is specific to a particular domain to which the medical imaging system corresponds.
(12) The apparatus of (11), wherein the processing circuitry is further configured to: obtain a second image dataset, and use the obtained second image dataset to train, as the pretrained second neural network, a feature extractor for extracting a feature specific to the particular domain.
(13) The apparatus of (12), wherein the processing circuitry is further configured to train the feature extractor by: iteratively alternating between training a generator included in a generative adversarial neural network and a discriminator included in the generative adversarial neural network, until a predetermined criterion is met, and using the trained discriminator as the pretrained second neural network.
(14) The apparatus of (13), wherein the discriminator includes one or more layers of a U-net.
(15) The apparatus of (11), wherein the processing circuitry is further configured to collect the first image dataset through a simulation, an experiment, and/or a clinical procedure within the particular domain.
(16) The apparatus of (12), wherein the processing circuitry is further configured to obtain the second image dataset through a simulation, an experiment, and/or a clinical procedure within the particular domain.
(17) The apparatus of (15), wherein the processing circuitry is further configured to use the collected first image dataset, or a subset of the collected first image dataset, as the obtained second image dataset.
(18) The apparatus of (11), wherein the loss function is a contrastive learning loss function, and the processing circuitry is further configured to train the first neural network by: obtaining, from the collected first image dataset, first image data, second image data, and third image data, and based on the contrastive learning loss function, using the first image data as input data, and the second and third image data as label data, to update a parameter of the first neural network, until a predetermined criterion is met.
(19) The apparatus of (11), wherein the loss function is a perceptual loss function, and the processing circuitry is further configured to train the first neural network by: obtaining, from the collected first image dataset, first image data and second image data, and based on the perceptual loss function, using the first image data as input data and the second image data as label data to update a parameter of the first neural network, until a predetermined criterion is met.
(20) A non-transitory computer readable medium having instructions stored therein that, when executed by one or more processors, cause the one or more processors to perform a method for performing image data processing in a medical imaging system, the method comprising: collecting a first image dataset; using the collected first image dataset, training a first neural network, based on a loss function having a perceptual component; and using the trained first neural network, inferring output image data from input image data obtained by the medical imaging system, such that the inferred output image data has an image quality better than an image quality of the obtained input image data, wherein the training of the first neural network uses a pretrained second neural network, and the pretrained second neural network is specific to a particular domain to which the medical imaging system corresponds.

Numerous modifications and variations of the embodiments presented herein are possible in light of the above teachings. It is therefore to be understood that within the scope of the claims, the disclosure may be practiced otherwise than as specifically described herein.

What is claimed IS:

1. A method for performing image data processing in a medical imaging system, the method comprising:
collecting a first image dataset;
using the collected first image dataset, training a first neural network, based on a loss function having a perceptual component; and
using the trained first neural network, inferring output image data from input image data obtained by the medical imaging system, such that the inferred output image data has an image quality better than an image quality of the obtained input image data,
wherein the training of the first neural network uses a pretrained second neural network, and the pretrained second neural network is specific to a particular domain to which the medical imaging system corresponds.

2. The method of claim 1, further comprising:
obtaining a second image dataset, and
using the obtained second image dataset to train, as the pretrained second neural network, a feature extractor for extracting a feature specific to the particular domain.

3. The method of claim 2, wherein the step of training the feature extractor further comprises:
iteratively alternating between training a generator included in a generative adversarial neural network and a discriminator included in the generative adversarial neural network, until a predetermined criterion is met, and
using the trained discriminator as the pretrained second neural network.

4. The method of claim 3, wherein the discriminator includes one or more layers of a U-net.

5. The method of claim 1, wherein the collecting step further comprises collecting the first image dataset through a simulation, an experiment, and/or a clinical procedure within the particular domain.

6. The method of claim 2, wherein the obtaining step further comprises obtaining the second image dataset through a simulation, an experiment, and/or a clinical procedure within the particular domain.

7. The method of claim 5, wherein the obtaining step further comprises using the collected first image dataset, or a subset of the collected first image dataset, as the obtained second image dataset.

8. The method of claim 1, wherein the loss function is a contrastive learning loss function, and the step of training the first neural network further comprises:
obtaining, from the collected first image dataset, first image data, second image data, and third image data, and
based on the contrastive learning loss function, using the first image data as input data, and the second and third image data as label data, to update a parameter of the first neural network, until a predetermined criterion is met.

9. The method of claim 1, wherein the loss function is a perceptual loss function, and the step of training the first neural network further comprises:
obtaining, from the collected first image dataset, first image data and second image data, and based on the perceptual loss function, using the first image data as input data and the second image data as label data to update a parameter of the first neural network, until a predetermined criterion is met.

10. The method of claim 1, wherein the particular domain is 2D projection X-ray imaging, Computed Tomography (CT) imaging, Magnetic Resonance Imaging (MRI), Positron Emission Tomography (PET) imaging, or ultrasound (US).

11. An apparatus for performing image data processing in a medical imaging system, the apparatus comprising:
processing circuitry configured to
collect a first image dataset,
using the collected first image dataset, train a first neural network, based on a loss function having a perceptual component, and
using the trained first neural network, infer output image data from input image data obtained by the medical imaging system, such that the inferred output image data has an image quality better than an image quality of the obtained input image data,
wherein the training of the first neural network uses a pretrained second neural network, and the pretrained second neural network is specific to a particular domain to which the medical imaging system corresponds.

12. The apparatus of claim 11, wherein the processing circuitry is further configured to:
obtain a second image dataset, and
use the obtained second image dataset to train, as the pretrained second neural network, a feature extractor for extracting a feature specific to the particular domain.

13. The apparatus of claim 12, wherein the processing circuitry is further configured to train the feature extractor by:
iteratively alternating between training a generator included in a generative adversarial neural network and a discriminator included in the generative adversarial neural network, until a predetermined criterion is met, and
using the trained discriminator as the pretrained second neural network.

14. The apparatus of claim 13, wherein the discriminator includes one or more layers of a U-net.

15. The apparatus of claim 11, wherein the processing circuitry is further configured to collect the first image dataset through a simulation, an experiment, and/or a clinical procedure within the particular domain.

16. The apparatus of claim 12, wherein the processing circuitry is further configured to obtain the second image dataset through a simulation, an experiment, and/or a clinical procedure within the particular domain.

17. The apparatus of claim 15, wherein the processing circuitry is further configured to use the collected first image dataset, or a subset of the collected first image dataset, as the obtained second image dataset.

18. The apparatus of claim 11, wherein the loss function is a contrastive learning loss function, and the processing circuitry is further configured to train the first neural network by:
obtaining, from the collected first image dataset, first image data, second image data, and third image data, and
based on the contrastive learning loss function, using the first image data as input data, and the second and third image data as label data, to update a parameter of the first neural network, until a predetermined criterion is met.

19. The apparatus of claim 11, wherein the loss function is a perceptual loss function, and the processing circuitry is further configured to train the first neural network by:
obtaining, from the collected first image dataset, first image data and second image data, and based on the perceptual loss function, using the first image data as input data and the second image data as label data to update a parameter of the first neural network, until a predetermined criterion is met.

20. A non-transitory computer readable medium having instructions stored therein that, when executed by one or more processors, cause the one or more processors to perform a method for performing image data processing in a medical imaging system, the method comprising:
collecting a first image dataset;
using the collected first image dataset, training a first neural network, based on a loss function having a perceptual component; and
using the trained first neural network, inferring output image data from input image data obtained by the medical imaging system, such that the inferred output image data has an image quality better than an image quality of the obtained input image data,
wherein the training of the first neural network uses a pretrained second neural network, and the pretrained second neural network is specific to a particular domain to which the medical imaging system corresponds.
